(19) United States
(12) Patent Application Publication
(10) Pub. No.: US 2012/0289613 A1
(43) Pub. Date: Nov. 15, 2012

(54) EMULSIONS, COMPOSITIONS AND DEVICES INCLUDING GRAPHENE OXIDE, AND METHODS FOR USING SAME

(75) Inventor: Jiaxing Huang, Wilmette, IL (US)

(73) Assignee: SONY CORPORATION, Tokyo (JP)

(21) Appl. No.: 13/467,590

(22) Filed: May 9, 2012

Related U.S. Application Data
(60) Provisional application No. 61/484,307, filed on May 10, 2011.

(51) Int. Cl. A61K 47/12; H01B 1/12; B01J 31/04; H01B 1/00; H01B 1/04; B82Y 30/00

(52) U.S. Cl. 514/784; 252/500; 252/510; 502/172; 977/779; 977/952; 977/950; 977/948

(57) ABSTRACT

Provided are an emulsion comprising graphene oxide, a first fluid and a second fluid, and a drug delivery system comprising the emulsion. This emulsion is based on the discovery that graphene oxide is an amphiphile with hydrophilic edges and a more hydrophobic basal plane, and thus graphene oxide can act as a surfactant. Since the degree of ionization of the edge —COOH groups of the graphene oxide is affected by pH, the amphiphilicity of graphene oxide can be adjusted based on pH. Therefore, a method of separating a first liquid from a second liquid by providing an emulsion comprising graphene oxide, the first liquid and the second liquid is also provided. It was also discovered that graphene oxide can act as a molecular dispersing agent to process insoluble materials. Based on this discovery, a composition comprising graphene oxide, a solvent and an insoluble solid is provided.

### CROSS REFERENCES TO RELATED APPLICATIONS

This application claims the benefit of U.S. patent application No. 61/484,307, filed on May 10, 2011, the entire contents of which are incorporated herein.

### BACKGROUND

The present application relates to graphene oxide compositions based on the discovery that graphene oxide is amphiphilic rather than hydrophilic. More particularly, the present invention relates to a graphene oxide emulsion, a drug delivery system comprising the emulsion, a method of separating a first liquid from a second liquid comprising providing a graphene oxide emulsion, and a composition comprising graphene oxide, a solvent and an insoluble solid.

A graphite oxide sheet (hereinafter referred to as graphene oxide (“GO”)) is the product of chemical exfoliation of graphite and has been known for more than a century. GO has been largely viewed as hydrophilic, presumably due to its excellent colloidal stability in water.

### SUMMARY

It has surprisingly been discovered that GO is an amphiphile with hydrophilic edges and a more hydrophobic basal plane. GO can thus act like a surfactant, as measured by its ability to adsorb on interfaces and lower the surface or interfacial tension. Since the degree of ionization of the edge —COOH groups is affected by pH, GO's amphiphilicity can also be adjusted based on pH. In addition, the size-dependent amphiphilicity of GO sheets was observed. Since each GO sheet is a single molecule as well as a colloidal particle, the molecule-colloid duality makes it behave like both a molecular and a colloidal surfactant. For example, GO is capable of creating highly stable Pickering emulsions of organic solvents like solid particles. It can also act as a molecular dispersing agent to process insoluble materials such as graphite and carbon nanotubes in water. The ease of its conversion to chemically modified graphene could enable new opportunities in solution processing of functional materials.

The present application provides in an embodiment a graphene oxide emulsion capable of forming emulsions of a first liquid such as toluene or oil in a second liquid such as water. Because the graphene oxide has the ability to form droplets of the first liquid, the graphene oxide may also be used to separate the first liquid from the second liquid. Further, a composition comprising graphene oxide, a solvent and an insoluble solid is provided in an embodiment. The insoluble solid may be dispersed in the solvent due to the ability of the graphene oxide to act as a surfactant.

According to an embodiment, there is provided an emulsion comprising: graphene oxide; a first fluid; and a second fluid. In the emulsion, the first fluid may be water, and the second fluid may be an organic solvent such as toluene. The amount of graphene oxide dispersed in the water ranges from 0.0095 mg to 0.95 mg per ml of water.

The emulsion may also comprise droplets of the organic solvent. In one embodiment, the droplets are submillimeter-sized. In another embodiment, the droplets have sizes ranging from 0.267 mm to 1.347 mm. The droplets may also be coated with graphene oxide.

According to another embodiment, there is provided a drug delivery system including an emulsion. The emulsion comprises graphene oxide, a first fluid, and a second fluid. The first fluid and/or the second fluid can comprise a drug molecule.

Another embodiment provides a method of separating a first liquid from a second liquid. The method comprises providing an emulsion including graphene oxide, the first liquid and the second liquid. The first liquid can be separated from the second liquid by adjusting the pH of the emulsion. The first liquid can be water, and the second liquid can be oil.

According to a further embodiment, there is provided a composition including graphene oxide, a solvent, and an insoluble solid. The solvent can be water. The insoluble solid may be, for example, graphite, single-walled carbon nanotubes, multi-walled carbon nanotubes or conducting polymer polyaniline powders. The composition can be used in a transparent conducting film, an electrode or a catalyst.

The transparent conducting film can include the composition deposited on a substrate. The graphene oxide in the transparent conducting film may be treated by a thermal treatment or a chemical treatment. The transparent conducting film may be used in a touch panel, an electrode of a dye sensitized solar cell, a display or an electrode of a light emitting device.

Additional features and advantages are described herein, and will be apparent from the following Detailed Description and the figures.

### BRIEF DESCRIPTION OF THE FIGURES

FIG. 1A is a schematic view showing the molecular structure of GO.

FIG. 1B is a schematic illustration showing the flotation of GO in carbonated water.

FIG. 1C is a graph showing the surface pressure for a flotation experiment in an LB trough.

FIG. 1D shows BAM images of a GO water surface before and after flotation.

FIG. 1E shows FQM images of GO sheets collected by dip coating before and after flotation.

FIG. 2A is a schematic illustration showing GO sheets deposited on a silicon wafer from a stock dispersion by drop casting.

FIG. 2B is a schematic illustration showing GO sheets deposited on a silicon wafer from a stock dispersion by LB assembly

FIG. 2C is a SEM image of the drop-casted sample of FIG. 2A.

FIG. 2D is a SEM image of a LB assembly sample obtained by dip coating from a surface.

FIG. 2E is a SEM image of a LB assembly sample obtained by dip coating from a subphase.

FIG. 3A shows a toluene/GO water mixture in which the concentration of GO is 0.95 mg/mL and a microscopy image of the toluene droplets.

FIG. 3B shows a toluene/GO water mixture in which the concentration of GO is 0.47 mg/mL and a microscopy image of the toluene droplets.

FIG. 3C shows a toluene/GO water mixture in which the concentration of GO is 0.19 mg/mL and a microscopy image of the toluene droplets.

FIG. 3D shows a toluene/GO water mixture in which the concentration of GO is 0.095 mg/mL and a microscopy image of the toluene droplets.

FIG. 3E shows a toluene/GO water mixture in which the concentration of GO is 0.047 mg/mL and a microscopy image of the toluene droplets.

FIG. 3F shows a toluene/GO water mixture in which the concentration of GO is 0.019 mg/mL and a microscopy image of the toluene droplets.

FIG. 3G shows a toluene/GO water mixture in which the concentration of GO is 0.0095 mg/mL and a microscopy image of the toluene droplets.

FIG. 3H shows an increase in droplet size.

FIG. 4A is a schematic illustration showing the reversible protonation of edge —COOH groups of a GO molecule.

FIG. 4B shows a toluene/GO water biphasic mixture at a pH of 10.

FIG. 4C shows a toluene/GO water biphasic mixture at a pH of 5.

FIG. 4D shows a toluene/GO water biphasic mixture at a pH of 2.

FIG. 4E shows a toluene/GO water biphasic mixture at a pH of 10.

FIG. 4F is a graph showing the interfacial tension between toluene and aqueous GO dispersions at various pH values.

FIG. 5A is an image showing graphite powder in GO water and DI water.

FIG. 5B is an optical microscopy image of untreated graphite powder.

FIG. 5C is a SEM image of a graphite powder sample sonicated in DI water.

FIG. 5D is a SEM image of a graphite powder sample sonicated in GO water.

FIG. 6A is an image showing multiwalled carbon nanotubes in GO water and DI water.

FIG. 6B is a graph showing the absorbance in the visible range over time of the multiwalled carbon nanotubes in GO water and the DI water.

FIG. 6C is a SEM image of a multiwalled carbon nanotube sample before sonication in DI water.

FIG. 6D is a SEM image of the multiwalled carbon nanotube sample of FIG. 6C after sonication in DI water.

FIG. 6E is a SEM image of a multiwalled carbon nanotube sample after sonication in GO water.

FIG. 6F is an AFM image of GO stabilized multiwalled carbon nanotubes deposited on a SiO₂/Si substrate.

FIG. 6G is a graph showing height profile data along the arrow shown in FIG. 6F.

FIG. 6H is a graph showing the sheet resistance of the multiwalled carbon nanotube/GO films.

### DETAILED DESCRIPTION

Embodiments of the present application will be described below in detail with reference to the drawings.

Surfactants are amphiphilic substances that can adsorb on interfaces and lower the surface or interfacial tension. Surfactants are used in numerous technologies such as detergents, emulsifiers, and dispersing agents. GO is the product of chemical exfoliation of graphite and has been known for more than a century. It is essentially a graphene sheet derivatized by carboxylic acid at the edges and phenol hydroxyl and epoxide groups mainly on the basal plane.

GO has conventionally been viewed as hydrophilic, presumably due to its excellent colloidal stability in water. However, it has surprisingly been discovered that GO is an amphiphile with hydrophilic edges and a more hydrophobic basal plane. GO can thus act like a surfactant, as measured by its ability to adsorb on interfaces and lower the surface or interfacial tension. Since the degree of ionization of the edge —COOH groups is affected by pH, GO's amphiphilicity can also be changed by adjusting the pH. GO can also act as a molecular dispersing agent to process insoluble solids such as graphite and carbon nanotubes in water.

Based on these discoveries, the present application provides in an embodiment an emulsion comprising GO, a first fluid and a second fluid. According to a further embodiment, a method of separating a first fluid and a second fluid using the GO emulsion is provided. In another embodiment, the present application provides a composition comprising GO, a solvent and an insoluble solid.

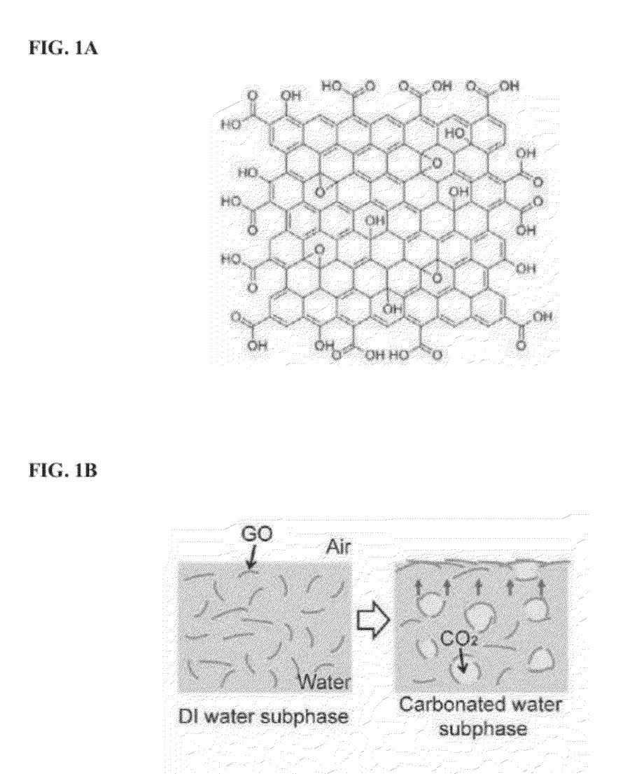

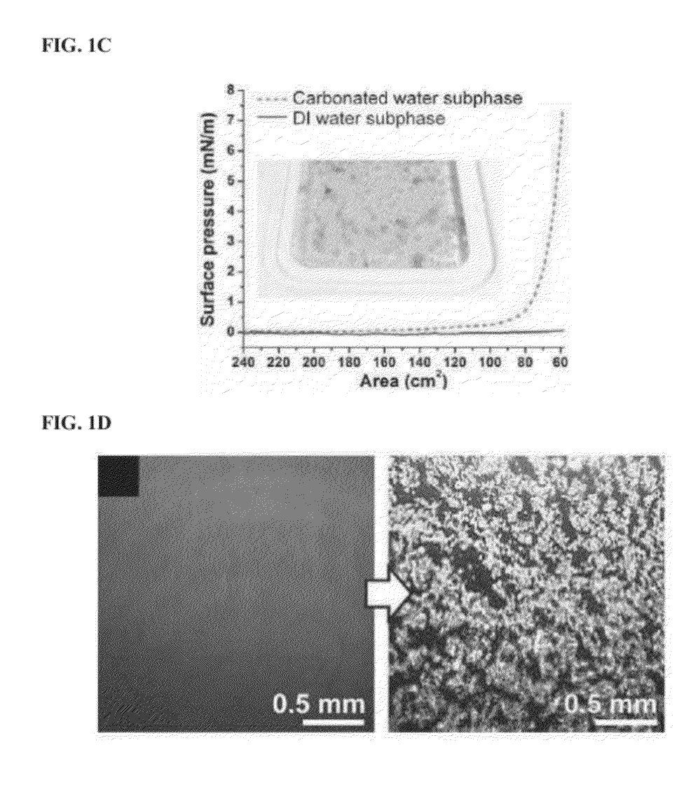

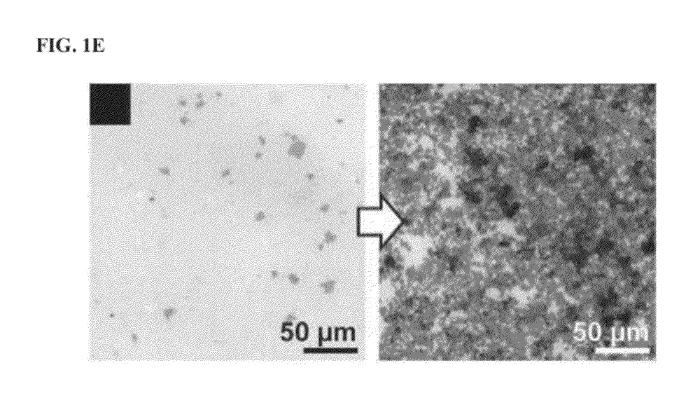

GO has been known to disperse well in water since its first discovery over a century ago and thus has been routinely described as hydrophilic in the literature. As shown in FIG. 1A, GO's water dispersibility and hydrophilicity have primarily been attributed to the ionizable edge-COOH groups. For example, GO can be viewed as a two-dimensional molecular amphiphile, with hydrophobic π domains interspersed on its basal plane and hydrophilic —COOH groups on the edges as shown in the structural model.

However, its basal plane is essentially a network of hydrophobic polyaromatic islands of unoxidized benzene rings. Therefore, GO should be viewed as an amphiphile with a largely hydrophobic basal plane and hydrophilic edges.

On the other hand, GO is characterized by two abruptly different length scales. While its thickness is determined by a single atomic layer, the lateral dimension extends up to tens of micrometers. Since GO has the characteristics of both a molecule and a colloidal particle, the key issues is whether GO will behave like a molecular amphiphile or a colloidal surfactant. To test the hypothesis, the activity of GO at air-water, liquid-liquid, and liquid-solid interfaces was studied.

GO was synthesized by a modified Hummers method from graphite powder (Bay Carbon, SP-1). For the CO₂ flotation experiment, GO was dispersed in commercially available carbonated water at a concentration of 0.01 mg/mL. A higher GO concentration hinders Brewster angle microscopy (“BAM”) observation of floating materials, as the GO sheets in the solution can generate a high-level background scattering. The experiment was carried out on a Langmuir-Blodgett (“LB”) trough (from Nima Technology) equipped with a tensiometer and a BAM (from Nima Technology). Fluorescence quenching microscopy (“FQM”) was performed as previously reported using fluorescein/polyvinylpyrrolidone (Mw=55,000 D) as the fluorescent layer.

To create a stock dispersion with polydispersed sizes, a heavily sonicated GO dispersion was mixed with an unsonicated GO dispersion. The size-dependent amphiphilicity of GO was tested at an air-water interface using an LB assembly. A small aliquot of the stock dispersion was spread onto the water surface from a water-methanol mixture. Dip coating was performed from either between or outside of the barriers to collect GO sheets floating on the water surface or in the subphase, respectively. For Pickering emulsion experiments, an organic solvent was mixed with GO water (i.e., GO dispersed in deionized water at 0.05 mg/mL) at half or equal volume and shaken by hand.

Generally, a decreased volume ratio of organic solvent to GO water produced better emulsions. Microscopy images of the emulsion droplets were taken directly through the horizontally placed vials with a Nikon SMZ-1500 stereoscope. The diameters of >100 randomly chosen droplets (>20 for very large ones) were measured. The pH value of GO water was modified by adding HCl (1 M) or NaOH (1 M) solution. The ζ potential was measured with Malvern Instruments' Zetasizer Nano system. Drop shape analysis was performed with a Krüss DSA 100 instrument by creating a drop of aqueous GO dispersion with a volume of ˜35 μL in toluene. The drop volume was held constant for 40 minutes before being reduced by about 30% at a rate of 2.5 μL/min.

For the solid dispersion experiments, graphite (Asbury, 3763) or carbon nanotubes (CNT, Strem Chemicals, multiwalled, diameter around 20 nm) powder was added into 10 mL of GO water at a mass ratio of 30:1 (graphite/GO) or 1:3 (CNT/GO), respectively. Then the dispersion was sonicated for 30 minutes using a Misonix S-4000 cup-horn ultrasonicator. A maximum amplitude of 80% was employed for graphite and 40% for CNT samples, respectively. After sonication, the supernatant was carefully collected and centrifugated at 1000 rpm for 5 min to remove nondispersed chunks. For optical absorbance measurements, the CNT-GO water was diluted five times and measured on a UV-vis spectrometer (Beckman, DU 520).

Scanning electron microscopy (“SEM”) images were taken on a Hitachi FE-SEM S-4800 instrument. Atomic force microscopy (“AFM”) images were acquired on a scanning probe microscope (Veeco, MultiMode V). CNT/GO thin films were prepared by vacuum filtration and transferred onto quartz substrates for sheet resistance measurement. To reduce GO, the film was thermally annealed at 200 or 400° C. for one hour in a muffle furnace. GO was also chemically reduced by exposing the film to hot hydrazine vapor (hydrazine monohydrate, Sigma Aldrich) at 200° C. for 1 hour. Sheet resistance was measured by a four-point probe setup.

In prior work, it was first discovered that GO can float on the water surface during LB assembly without the need for structural modification or extra surfactant. This suggests that GO should be surface active, just like molecular amphiphiles. If this is true, the surface of GO water should be covered with a layer of sheets, which can be directly observed by BAMsa surface selective imaging technique. As shown in FIG. 1D (left), BAM observation of freshly prepared GO water revealed little surface-active material. However, GO sheets started to appear after a few hours. This is attributed to the slow diffusion of GO sheets, which are typically micrometer-sized, to the surface due to their large “molecular” mass. To accelerate their migration to the surface, a flotation process was designed using commercially available carbonated water (FIG. 1B, right). As shown in FIG. 1B, GO is first captured by the rising CO₂ bubbles and then transported to the water surface.

Boiling stones were added to release the solvated CO₂ (FIG. 1C, inset). If GO sheets are indeed surface-active, they would adhere to the rising CO₂ bubbles and become thermodynamically trapped after they reach the air-water interface to minimize the surface energy (i.e., lower the surface tension).

The experiment was carried out in an LB trough equipped with a tensiometer to monitor the surface tension and a BAM to watch the surface. To facilitate the observation, the floating materials were concentrated by compressing the water surface using barriers. In a control experiment, the surface pressure of GO water, which is essentially a measure of decreased surface tension, remained unchanged even after full compression (FIG. 1C, solid line). However, after flotation, an increased surface pressure during isothermal compression was clearly observed (FIG. 1C, dashed line), thus indicating the presence of GO at the water surface. Meanwhile, BAM revealed a large amount of material on the surface right after the evolution of bubbles (FIG. 1D, right). The floating materials were collected on a glass substrate, which can be conveniently imaged by an FQM technique that was recently developed.

FIG. 1E confirms the much increased surface density of GO sheets after flotation. Flotation can be achieved with other gases such as nitrogen and air through DI water. Upward convection flows induced by heating or evaporation were also found to accelerate the surface enrichment of GO sheets, as revealed by BAM observation. Both the in situ BAM images of the water surface (FIG. 1D) and the FQM images of GO sheets collected by dip coating (FIG. 1E) show a massive increase of GO at the surface after flotation.

The surface activity of GO thus confirms that it is indeed amphiphilic. This new insight is important for understanding the processing and assembly of GO-based materials. For example, now it is clear why GO tends to form a thin film at the water surface during evaporation.

The hypothesis that GO is an amphiphile with a largely hydrophobic basal plane and hydrophilic edges implies that its amphiphilicity should be size dependent. As the size decreases, the edge-to-area ratio would increase. Therefore, smaller sheets should be more hydrophilic due to higher charge density resulting from the ionizable edge —COOH groups.

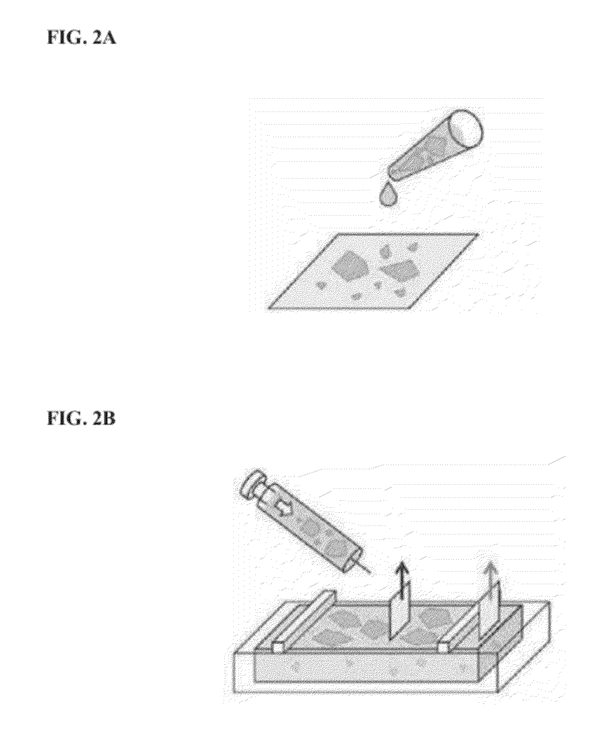

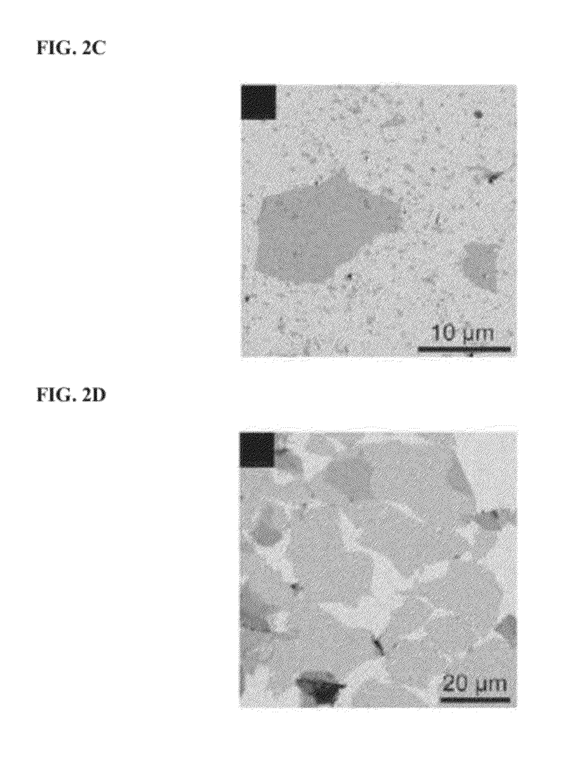

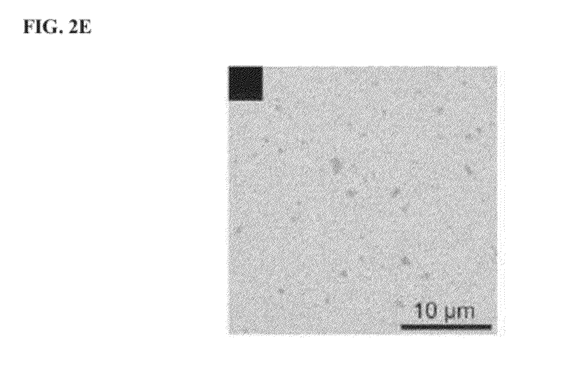

To test this idea, GO water was heavily sonicated to reduce the size of GO sheets. Indeed, increased ζ potential of the GO dispersion was observed after sonication. The dispersion of smaller GO sheets was then mixed with an unsonicated sample to create a new stock dispersion. The GO sheets were then deposited on a Si wafer by drop casting (FIG. 2A) and imaged to evaluate their sizes. The SEM image in FIG. 2C reveals both large (>5 μm) and small (≦1 μm) GO pieces in the stock dispersion. The stock dispersion was then spread onto the air-water interface for LB assembly. If GO has size-dependent amphiphilicity, larger sheets should float on the water surface while smaller ones could sink due to increased hydrophilicity. This was indeed observed.

As shown in FIG. 2B, GO sheets floating on the surface were collected by dip coating from the area between the two barriers, while those in the subphase were collected by dip coating from the area outside the two barriers. SEM images (FIGS. 2D, 2E) of samples thus collected clearly show that spontaneous size separation did occur during LB assembly. The air-water interface had effectively acted as a filter to support the large sheets on the surface (FIG. 2D) while sinking the small pieces into the subphase (FIG. 2E). This implies that smaller GO pieces are more hydrophilic

The density of GO sheets collected from the subphase was low, due to much lower GO concentration in the bulk of the subphase than on the surface. The results support the hypothesis that GO becomes more hydrophilic as its size decreases, which could be used to design methods of size separation as demonstrated in FIG. 2. It is also quite intriguing that the water surface itself acts as a size-separation filter for GO sheets, which could be potentially extended to other colloid systems.

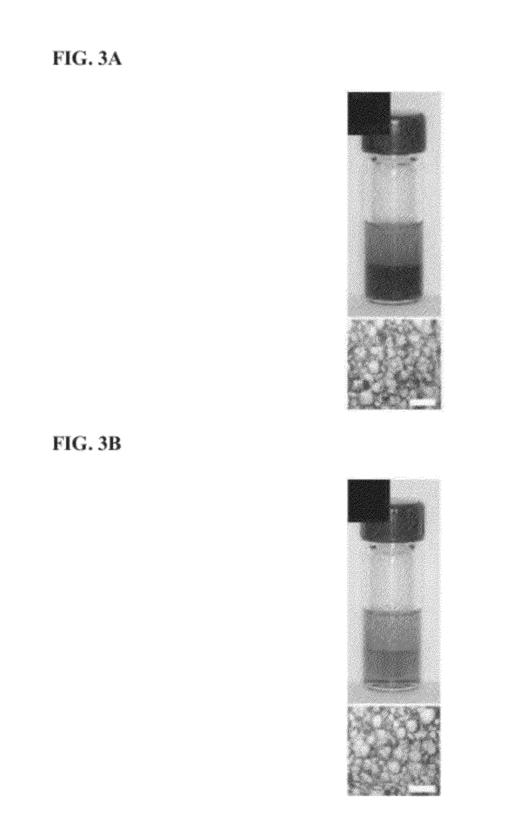

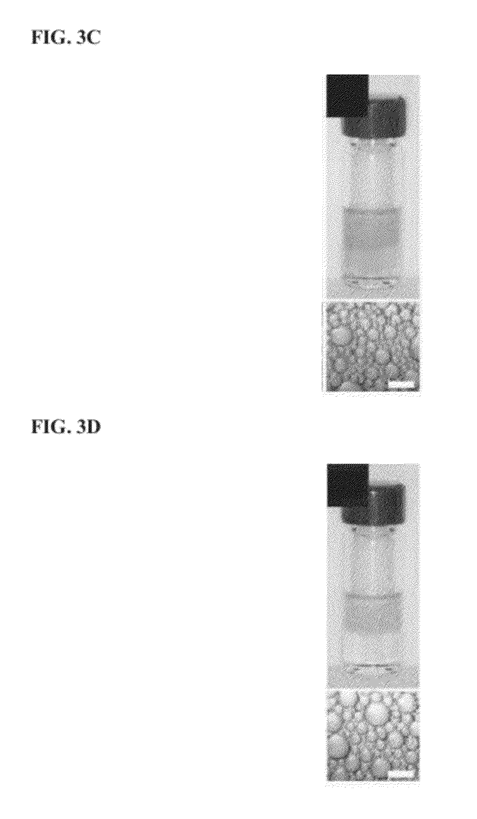

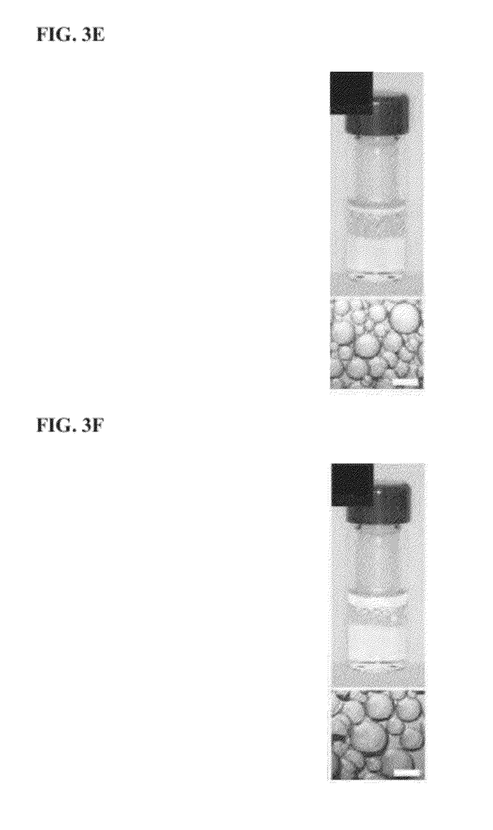

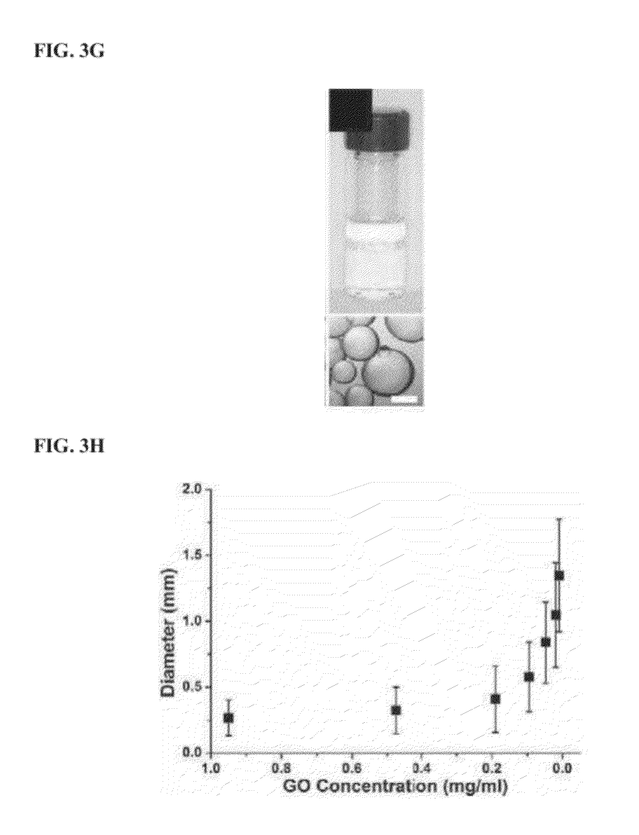

FIG. 3 demonstrates that GO can act as an emulsifier to create submillimeter-sized organic solvent droplets (e.g., toluene) that are stable in water for months. This is characteristic of particle stabilized Pickering emulsions, suggesting that GO is acting like a colloidal surfactant. The size of the toluene droplets was found to depend on the concentration of GO water. FIGS. 3A-G show that as the GO concentration is reduced, the volume of the emulsion phase is decreased. Meanwhile, FIG. 3H shows that the average sizes of the droplets increased from 0.267 mm (FIG. 3A) to 0.323 mm (FIG. 3B), 0.409 mm (FIG. 1C), 0.578 mm (FIG. 1D), 0.838 mm (FIG. 1E), 1.047 mm (FIG. 1F), and 1.347 mm in diameter (FIG. 1G), which is consistent with Pickering emulsions stabilized by colloidal particles.

Although the submillimeter-sized toluene droplets shown in FIGS. 3A-3D are much larger than the typical Pickering emulsions stabilized by colloidal particles (e.g., silica), they were remarkably stable against coalescence due to the high surface area of GO, which allows them to be kinetically trapped at the interface. The areas of GO sheets used in our experiments are typically in the range of hundreds to thousands of square micrometers, which are many orders of magnitude higher than the cross-sectional areas of typical colloidal particles.

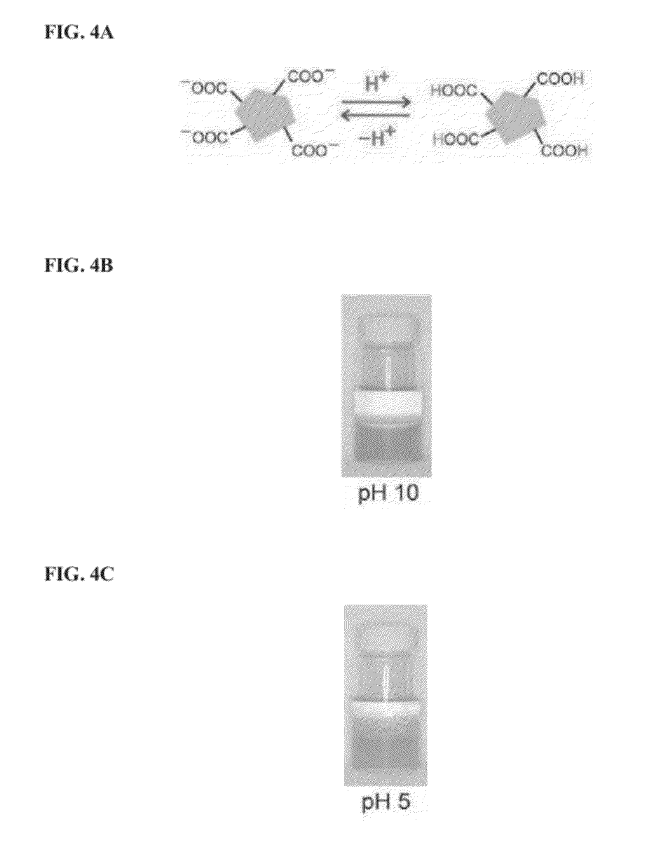

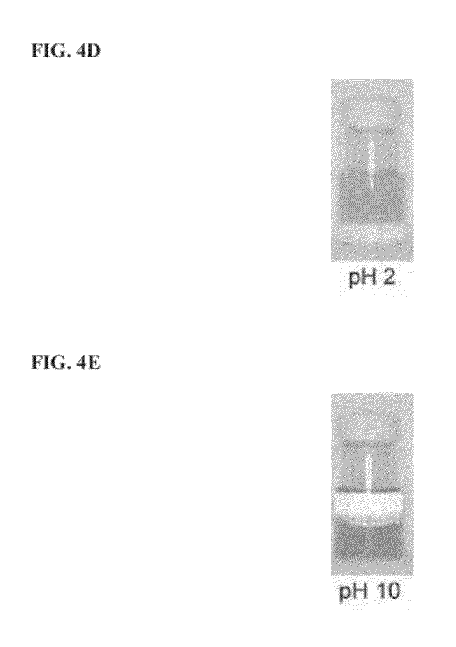

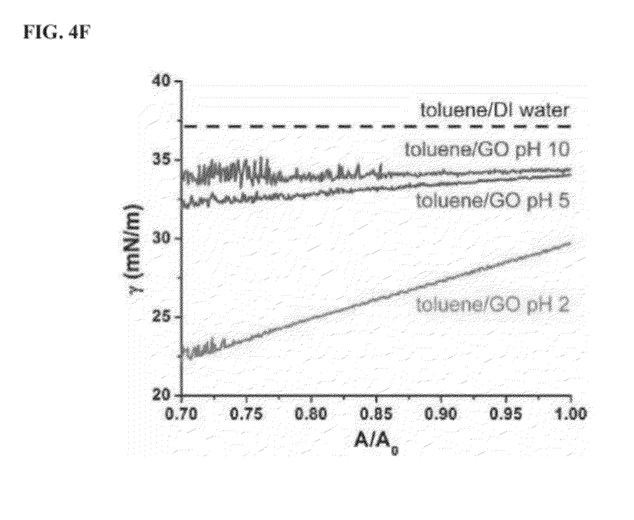

The amphiphilicity of GO can be controlled by changing the pH, as it affects the degree of ionization of the edge —COOH groups. For example, high pH values promote the deprotonation of the —COOH groups as shown in FIG. 4A, which would make GO more charged. In fact, the ζ potentials of GO water were measured to be −50.2 mV at pH 10 and −22.7 mV at pH 2, respectively, which were consistent with a prior report in literature. Therefore, GO sheets should become more hydrophilic as the pH is increased.

Indeed, when the pH was changed to 10 to form a basic solution as shown in FIG. 4B, GO is deprotonated, charged and more hydrophilic and was found to stay in the water phase such that no Pickering emulsions were created even after vigorous shaking However, as the pH was decreased and the solutions became more acidic, GO-coated toluene droplets started to form. This is because GO becomes more protonated, less charged and more hydrophobic as the pH is lowered.

FIG. 4C shows the emulsion phase obtained at around pH 5. In comparison to FIG. 4B, the color of the water phase was paler, since some GO was transferred to the emulsion phase. When the pH was lowered to 2 as shown in FIG. 4D, nearly all the GO was extracted, leaving the water phase clear of color. Meanwhile, the emulsion phase reached its maximum volume. When the pH was adjusted back to 10 as shown in FIG. 4E, the droplets coalesced into a continuous phase, ejecting GO back to water. Therefore, GO can be reversibly shuttled between water and the emulsion phase, which could make it useful for extraction or phase transfer applications.

The pH-dependent activity of GO was confirmed by drop shape analysis of the interfacial tension between GO water and toluene. FIG. 4F shows that a decrease in interfacial tension during compression was observed at all pH values but was much more pronounced for the acidic GO dispersions. The data in FIG. 4F was obtained by shrinking a suspended aqueous droplet so that the overall interfacial area decreases from an initial value of A0 to a lower value of A. A decrease in interfacial tension was observed for all pH values but became more pronounced at lower pH, confirming the pH-dependent amphiphilicity of GO.

In control experiments, the GO water was filtered once more and redispersed in DI water. Drop shape analysis showed that the reduction of interfacial tension by the filtrate was not as significant as that induced by the purified GO. Therefore, GO indeed acted as a surfactant for the oil-water system, as measured by its ability to adsorb at the oil-water interface (as shown in FIG. 4D) and reduce the interfacial tension (as shown in FIG. 4F). It was also found that GO can stabilize aromatic solvents more efficiently than aliphatic solvents, presumably due to stronger π-π interactions.

Although the reduction in interfacial tension was modest, Pickering emulsions in GO water appeared stable for an extended period of time (at least months). The large surface areas of the GO sheets can help them to be kinetically trapped at the interface, rendering long-term emulsion stability, if they indeed adopt the extended, flat-sheet geometry at the interface. The morphology of GO sheets on the droplet was indirectly examined by transferring them to a substrate. This was done by dip coating from the emulsion phase. When the substrate was in contact with the oil droplets, it tended to break the oil droplets and “peel off” the interfacial GO sheets, in a way similar to contact transfer or Langmuir-Shaffer deposition.

FQM imaging revealed that although there were many multilayer islands, the underlying layer was largely a monolayer of flat GO sheets. This implies that a monolayer of GO is sufficient to stabilize the oil-water interface. The multilayer domains were likely due to secondary deposition from collapsing droplets as they were broken by the substrate. Since GO is much enriched at the oil-water interfaces, dip coating from the emulsion always produces films much denser than those from the original GO water, which turns out to be a facile method for making GO films with high coverage.

One of the major applications of surfactants is as dispersing agents for the solution processing of solids. Inspired by the surfactant behaviors of GO at the air-water and liquid-liquid interfaces, the solid-liquid interface was tested to see if GO could act as a molecular dispersing agent. As a proof of concept, graphite and CNTs were chosen as the model system, both of which are known to be difficult to process in water. Since GO has many π-conjugated aromatic domains in its basal plane, it should be able to strongly interact with the surface of graphite and CNTs through π-π attractions. Some earlier reports also showed that GO was capable of adsorbing drug or dye molecules through π-π interactions. Therefore, the excellent water processability of GO could be inherited by forming complexes with graphite particles or CNTs.

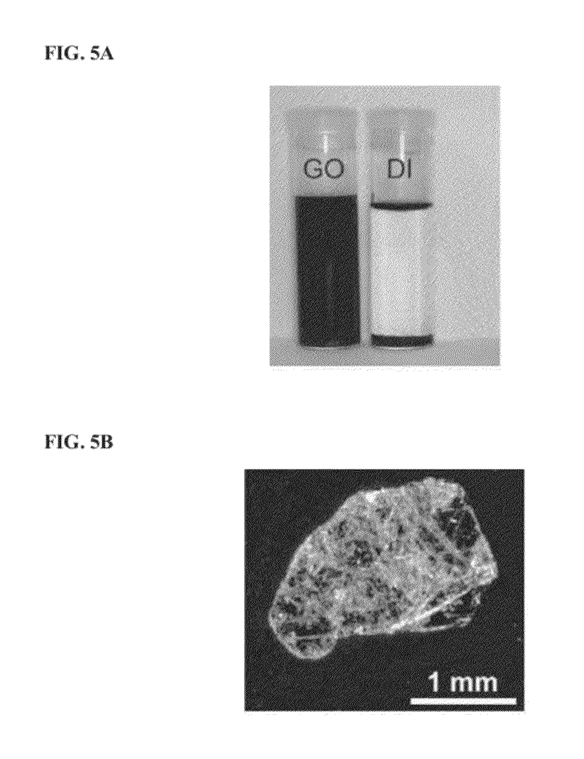

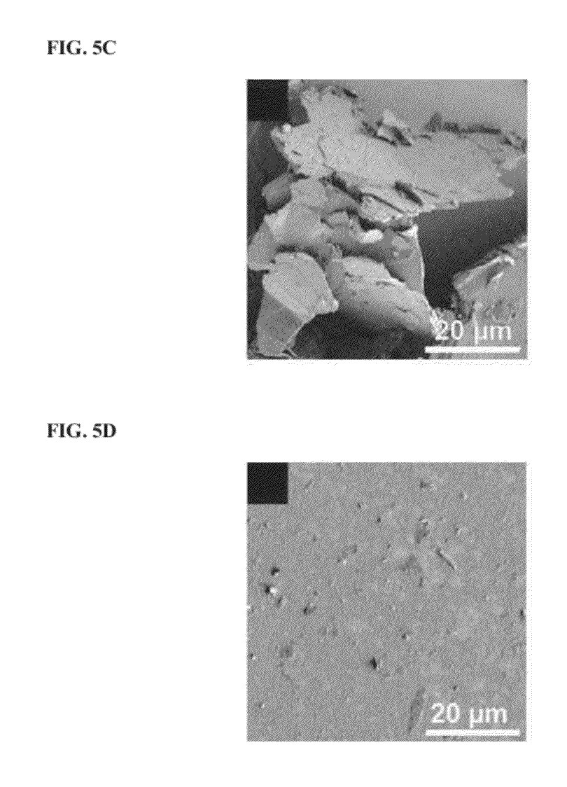

FIG. 5A shows that GO can effectively disperse graphite powders in water. For example, FIG. 5A shows that the graphite powder forms into a stable colloidal dispersion in GO water, whereas GO does not disperse at all in DI water. The starting powders were hundreds of micrometers to millimeters in diameter as shown in FIG. 5B. After being sonicated in water, they broke into thinner pieces of tens of micrometers as shown in FIG. 5C but still settled down right afterward as shown in FIG. 5A, right. However, FIG. 5D shows that in GO water, much smaller particles were obtained with diameters of only a few micrometers. This represents a reduction in size of nearly three orders of magnitude. In addition, the particles were found to be covered by GO sheets. The graphite dispersion in GO water stayed stable for days. Even though a large portion of the suspended particles eventually settled down, the suspended particles could be readily redispersed by gentle shaking or mild sonication.

The greater size reduction in GO water is likely a result of surface functionalization by GO, which makes the graphite particles better suspended and more effectively sonicated. On the other hand, the presence of GO sheets should greatly retard the motion of graphite particles in water during sonication. Therefore, when ultrasound induced microjets impinge on the particles, their kinetic energies can be better directed to break the particles.

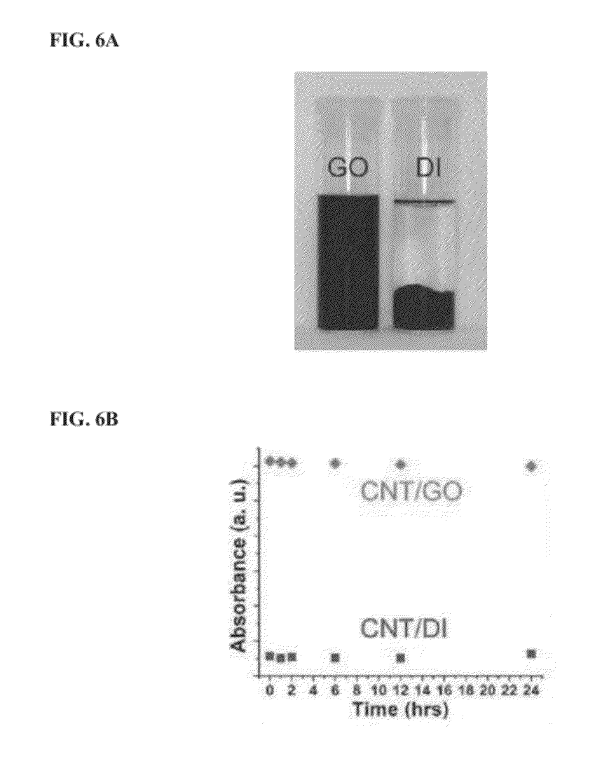

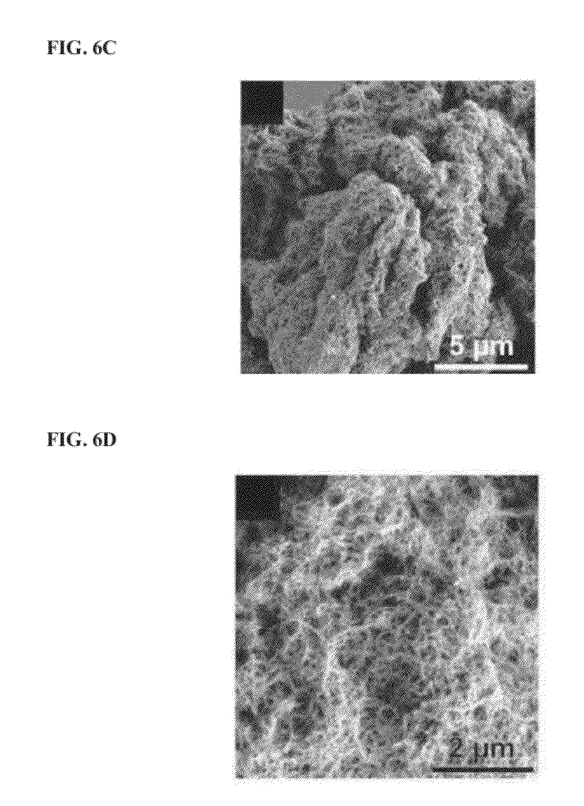

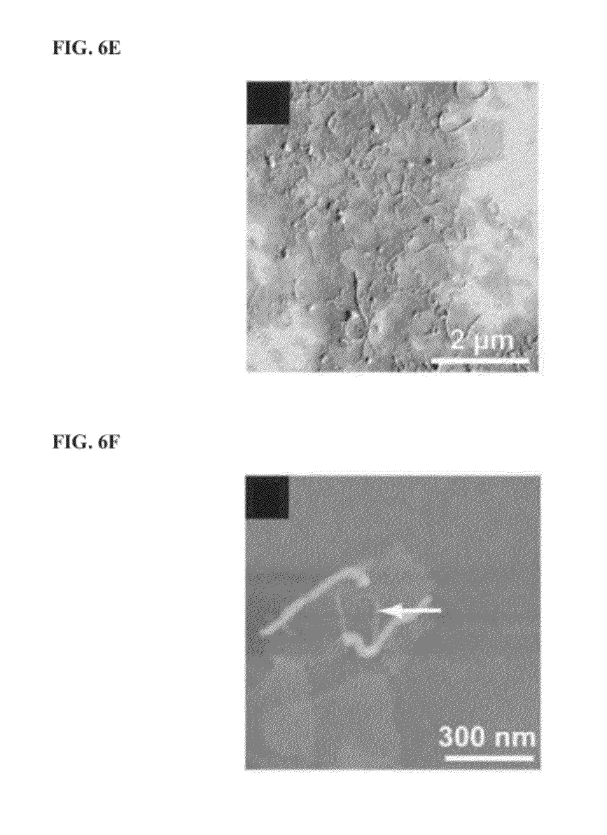

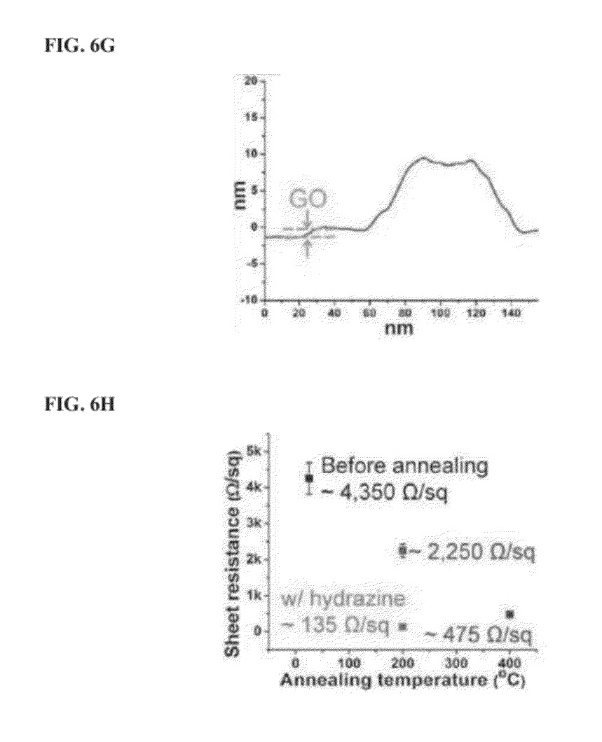

A tremendous amount of effort has been devoted to making CNTs water processable through wrapping by water-soluble materials. Since many surfactants for dispersing CNTs have polyaromatic components (e.g., pyrene35), GO should be able to adhere to CNTs and disperse them in water as well. FIG. 6A shows that CNTs indeed dispersed well in GO water with a 1:3 mass ratio after sonication. As is the case with graphite, sonication alone does not disperse CNTs in water. Instead, CNTs rapidly aggregate in DI water. As shown in FIG. 6B, the colloidal stability of the CNT-GO water was monitored by its optical absorbance over a period of 24 hours, which remained nearly constant after sonication. The dispersion was found to be stable for at least a few months. The absorbance of the supernatant of a CNT/DI water sample was negligible, which is consistent with the poor dispersibility of CNT in water.

Microscopy analysis as shown in FIG. 6C revealed that the initial CNTs samples were heavily entangled, which remained largely unaffected by sonication in water as shown in FIG. 6D. In contrast, CNTs sonicated in GO water were completely disentangled. Extensive microscopy observations by SEM (as shown in FIG. 6E) and AFM (as shown in FIGS. 6F-6G) revealed that almost all the CNTs in the sample were well dispersed, disentangled and adhered to GO, which is consistent with our hypothesis. Although the CNTs shown in FIG. 6 were multiwalled, it was found that GO can also effectively disperse single-walled CNTs in water.

GO can improve the dispersal of other π-conjugated materials such as conducting polymer polyaniline powders. Since it can be readily reduced to conductive, chemically modified graphene, GO could be a particularly attractive dispersing agent for solution processing of materials for electronic applications, since now the surfactant itself is a functional component as well.

Commonly used dispersing agents such as molecular surfactants, polymers, and DNA are usually insulating materials, which need to be removed afterward to avoid decreased conductivity. However, GO can actually provide more conducting pathways in the final complex after it is reduced, for example, by thermal or chemical treatment. FIG. 6H shows that the sheet resistance of a vacuum-filtered GO-CNT film indeed decreased significantly after either hydrazine vapor treatment or thermal annealing. The GO-CNT film can be made after depositing the CNT dispersed in GO water on a substrate and then treating the dispersion with thermal or chemical treatments. The GO-CNT film can be used in a touch panel, an electrode of a dye sensitized solar cell, a display or an electrode of a light emitting device.

During fabrication of electrodes in various electrochemistry devices, GO can act as a surfactant to disperse an electrode active material. For example, after it is deposited on a substrate, the GO or reduced GO can exist as a conducting adhesive agent or a conducting matrix of an active material. The GO-active material dispersion can be used as a cathode and/or anode of a primary battery, a rechargeable battery, a solar cell, a fuel cell, a capacitor, a sensor or an electrolysis electrode. The GO-active material dispersion can also act as both a catalyst and an electrode in a fuel cell or an air battery.

In conclusion, despite its excellent dispersibility in water, GO is an amphiphile that can adsorb onto interfaces and lower surface and interfacial tension. Its amphiphilicity can be adjusted by changing pH as it shuttles between water and the oil-water interface. Size-dependent amphiphilicity was also observed, leading to spontaneous interfacial size separation. GO is essentially a single atomic sheet, while its lateral dimension extends to the size of colloidal particles, which renders it a unique material exhibiting molecule-colloid duality. It creates highly stable Pickering emulsions of organic solvents like colloidal particles and disperses insoluble solids in water like molecular surfactants. This new insight echoes our earlier view that GO is an unconventional soft material. It should help to better understand and improve the solution processing of GO-based graphene materials and open up opportunities to design new functional GO-based hybrid materials.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. An emulsion comprising:
graphene oxide;
a first fluid; and
a second fluid.

2. The emulsion according to claim 1, wherein the first fluid comprises water and the second fluid comprises an organic solvent.

3. The emulsion according to claim 2, wherein the organic solvent is toluene.

4. The emulsion according to claim 2, wherein an amount of graphene oxide dispersed in the water ranges from 0.0095 mg to 0.95 mg per ml of water.

5. The emulsion according to claim 2, wherein the emulsion comprises droplets of the organic solvent.

6. The emulsion according to claim 5, wherein the droplets are submillimeter-sized.

7. The emulsion according to claim 5, wherein the droplets have sizes ranging from 0.267 mm to 1.347 mm.

8. The emulsion according to claim 5, wherein the droplets are coated with graphene oxide.

9. A drug delivery system including an emulsion, the emulsion comprising:
graphene oxide;
a first fluid; and
a second fluid.

10. The drug delivery system according to claim 9, wherein one of the first fluid and the second fluid comprises a drug molecule.

11. A method of separating a first liquid from a second liquid in a solution using graphene oxide, the method comprising:
adding graphene oxide to the solution; and
forming an emulsion comprising: graphene oxide, the first liquid and the second liquid, thereby separating the first liquid from the second liquid.

12. The method according to claim 11, further comprising adjusting the pH of the emulsion.

13. The method according to claim 11, wherein the first liquid comprises water and the second liquid comprises oil.

14. A composition comprising:
graphene oxide;
a solvent; and
an insoluble solid.

15. The composition according to claim 14, wherein the solvent comprises water.

16. The composition according to claim 14, wherein the insoluble solid is selected from the group consisting of graphite, carbon nanotubes, an electrode active material, catalysts, and conducting polymer polyaniline powders.

17. The composition according to claim 16, wherein the carbon nanotubes are selected from the group consisting of single-walled carbon nanotubes and multi-walled carbon nanotubes.

18. A transparent conducting film comprising the composition according to claim 16.

19. The transparent conducting film according to claim 18, wherein the composition is deposited on a substrate.

20. The transparent conducting film according to claim 18, wherein the graphene oxide is treated by any one of a thermal treatment an optical treatment, a laser treatment, and a chemical treatment.

21. A device comprising the transparent conducting film according to claim 18, wherein the device is selected from the group consisting of: a touch panel, an electrode in a dye sensitized solar cell, a display and a light emitting device electrode.

22. An electrode comprising the composition according to claim 16.

23. (canceled)

24. The electrode according to claim 22, wherein the graphene oxide is treated by any one of a thermal treatment, an optical treatment, a laser treatment, a chemical treatment.

25. A catalyst comprising the composition according to claim 16.

26. The catalyst according to claim 25, wherein the graphene oxide is treated by any one of a thermal treatment, an optical treatment, a laser treatment, and a chemical treatment.
